(19) United States
(12) Patent Application Publication
(10) Pub. No.: US 2015/0306237 A1
(43) Pub. Date: Oct. 29, 2015

(54) HYALURONIC ACID-ANTIOXIDANT CONJUGATES AND THEIR USES

(71) Applicant: UNIVERSITY OF GENEVA, Geneva 4 (CH)

(72) Inventors: Robert Gurny, Geneva (CH); Sema Gwendolyn Kaderli, Bernex (CH); Leonardo Scapozza, Grens (CH); Michael Moeller, Arzier (CH)

(21) Appl. No.: 14/653,637

(22) PCT Filed: Dec. 18, 2013
(86) PCT No.: PCT/EP2013/003842

§ 371 Date: Jun. 18, 2015

Related U.S. Application Data
(60) Provisional application No. 61/739,047, filed on Dec. 19, 2012.

(51) Int. Cl. A61K 47/48; A61K 31/728

(52) U.S. Cl. CPC A61K 47/48092; A61K 31/728

(57) ABSTRACT

The invention relates to hyaluronic acid (HA) compositions and HA conjugates comprising antioxidant compounds, pharmaceutical compositions thereof and their use in medical applications.

The present invention relates to compositions and pharmaceutical compositions comprising hyaluronic acids and antioxidants, conjugates thereof, and their use in medical and cosmetic applications.

### BACKGROUND OF THE INVENTION

Hyaluronic acid (HA) and compositions comprising HA have been known for a long time. HA has been combined with various other compounds and applied in a number of medical applications. In gel formulations, such applications include treatment of joint problems, and particularly for knee joint problems.

An approved medical treatment is e.g. Synvisc-One™ (hylan G-F 20) of Genzyme Corporation indicated for the treatment of pain in osteoarthritis (OA) of the knee in patients.

Eighty percent of the people above 75 years are affected by osteoarthritis (OA) [1]. One of the current therapies for OA is intra-articular injection of very high molecular weight hyaluronic acid (HA) (so called viscosupplementation) [2]. Therapy includes regular re-administration to replace HA which is degraded in vivo by inflammatory factors such as enzymes, immune cells and oxidant substances [1]. Because the articular retention time of injected HA formulations is limited [3], 3-4 injections are needed every 6 months.

In order to reduce the frequency of injections and to enhance the effect and efficacy of the treatment, an increased local residence time of the formulation would be desirable.

Thus it is an object of the invention to provide HA compounds, compositions and conjugates with increased retention times and improved methods useful in medical and/or in cosmetic applications, or at least to diminish the disadvantages of the prior art compounds and compositions and methods or to essentially reduce the disadvantages of the known techniques.

### SUMMARY OF THE INVENTION

In one aspect the invention relates to hyaluronic acid (HA) compositions comprising antioxidant compounds (Ax).

In another aspect the invention relates to hyaluronic acid conjugates comprising antioxidant compounds or consisting of HA and an Ax.

In another aspect the invention relates to the preparation of HA-Ax conjugates and additionally the in-vivo crosslinking of said compounds with each other engendering a cross-linked stabilized HA complex at the site of administration.

In another aspect the invention relates to pharmaceutical compositions of said compositions.

In another aspect the invention relates to medical applications and the use of the compositions and pharmaceutical compositions.

In yet another aspect the invention relates to an improved method of making HA-Ax conjugates.

### DETAILED DESCRIPTION OF THE INVENTION

The invention will be described in more detail below including preferred embodiments of the invention.

The invention relates to a composition comprising or consisting of a hyaluronic acid and an antioxidant.

In particular the invention is a composition comprising or consisting of a hyaluronic acid and an antioxidant wherein the hyaluronic acid is conjugated with an antioxidant.

Furthermore, the invention is a composition comprising or consisting of a hyaluronic acid and an antioxidant wherein the hyaluronic acid is conjugated with an antioxidant and/or wherein the hyaluronic acid conjugates are crosslinked with each other.

In a particular embodiment the invention is a composition comprising or consisting of a hyaluronic acid and an antioxidant wherein the hyaluronic acid is conjugated with an antioxidant and/or wherein the hyaluronic acid conjugates are crosslinked with each other and preferably wherein the crosslinking occurs in vivo.

In a preferred embodiment the composition according to the invention is characterized in that the hyaluronic acid antioxidant conjugate has one or more moieties which can crosslink in an oxidative system.

It is preferred that the hyaluronic acid antioxidant conjugate has one or more moieties which can crosslink in an oxidative system and whereby viscosification is engendered.

In another preferred embodiment the composition according to the invention the antioxidant is selected from the group consisting of aniline, 5-aminosylicylic acid, aminomethylcoumarine, 4-aminoresorcinol and ethylester cystein.

The invention can advantageously achieve useful viscosities which are preferably from 0.01 Pa·s to 100 Pa·s, more preferably 0.1 to 10 Pa·s. In preferred embodiments the composition is characterized by a viscosity of 0.05-10 Pa·s, preferably 0.5 to 5 Pa·s, more preferably 5 Pa·s as measured by rotational rheometry with a 35 mm diameter/2° cone-plate geometry at a 46 s-1 shear rate. For intra-articular application a wide range of typical viscosities ranging from 0.05 to 50 Pa·s may be used. Similar viscosities may be used for ophthalmic applications. Topical application can also be performed with highly viscous—several 100's Pa·s—or viscoelastic formulations.

Specifically for viscosupplementation in articular application preferred ranges are from 0.1 Pa s to 100 Pa s, preferably 0.1 Pa·s to 10 Pas. Current commercial viscosupplementation products have formulated pre-administration viscosities of from about 0.6 to 200 Pa·s. The compositions of the invention can be applied in such viscosity ranges as well. The advantage of the inventive compositions is however, that all such products are potentially losing viscosity as soon as they are injected due to degradation. So it is a trade off having the highest starting viscosity—that can be injected—to give the longest in vivo action in prior art products. The new and inventive compounds provided here are not as sensitive to this trade off, due to (i) resistance to (oxidative) degradation reducing viscosity loss; and (ii) in-vivo cross linking enhancing viscosity after administration.

The compositions of the invention thus exhibit an improved efficacy over a longer time period, enabling a longer beneficial effect from single administrations and a concomitant reduction in the frequency of necessary treatments. Preferably, the effective duration from a single administration should be one month, more preferably three months, and even more preferably 6 months or more.

Advantageously the compositions according to the invention are biocompatible. Preferably they have a biocompatibility comparable to unconjugated hyaluronic acid.

Also the composition according to the invention is not significantly more toxic than unconjugated hyaluronic acid per se.

The compositions as described above can be used in medical or cosmetic applications. In particular the compositions are useful in treating an ophthalmic, topical, systemic or intra-articular disease or disorder. Preferably the compositions according to the invention can be used for treating arthritis, joint pathologies, articular diseases, eye pathologies, as a skin treatment, or for tissue regeneration.

In one preferred embodiment the invention related to methods of treating joint pathologies, articular diseases, eye pathologies, skin treatment, tissue regeneration applying a composition of the invention to a subject in need thereof.

The compositions of the invention can be administered as is appropriate under the circumstances or with regard to the respective application, preferred a topical, i.v., i.m., s.c. administration, or administration into a joint or into an eye by intra-articular or intra-ocular injection.

The compositions of the invention can be used as such or in combination with a delivery means or/and as a medical device.

The compositions or pharmaceutical compositions according to the invention can be applied for use in aesthetic applications, preferably for cosmetic applications.

The compositions of the can be applied alone or in combination with any suitable adjuvants intradermally or subdermally.

In summary, the inventors have developed a novel strategy to protect the hyaluronic acid from the oxidation by covalent grafting of antioxidant moieties

In addition the inventors provide an improved HA-antioxidant coupling method by ethyl(dimethylaminopropyl)carbodiimide (EDC) in water has been first optimized with aniline, on the base of an existing protocol applied with 5ASA [4].

Five preferred antioxidants with single ring aromatic amide are selected as well as an amino methylcoumarin derivative with two aromatic rings. Tyramin is selected because of its linear amine and aromatic phenol and cysteine because of the presence of a free cysteine. Indeed, sulphur atoms are a sensor for reactive oxygen species and nitrogen that can alter lipids, proteins or DNA in the cells and this increases its stability and leads to a decreased accessibility to enzyme and increased resistance to physical shocks [5, 6]

Seven hyaluronic acid conjugates were prepared and characterized by ¹H-NMR; 2-aminophenol (2AP), 4-aminosalicylic acid (4ASA), 5-aminosalicylic acid (5ASA), 4-aminoresorcinol (4AR), methylcoumarin amino derivative (AMC, prepared according to [7]), ethylester cysteine (EECyst) and tyramin (Tyr). Indeed, the reactions of amide formation in water with EDC are poorly favorable because sensitive to pH and reaction time leading to formation of insoluble N-acyl-urea byproducts, causing insolubility of the polymer obtained [8].

The inventors have also evaluated the in vivo behavior of such products and their biocompatibility and toxicity and have surprisingly found that these compounds compared to hyaluronic acid as such do not exhibit significantly increased toxicity and they have found that these compounds are advantageously biocompatible.

Biocompatibility can be proven by clinical, macroscopic and histological evaluations. In particular HA-4Ar has shown superior results.

Another advantage of the inventive compositions is that they can be formed by synthesis but also that conjugates may cross-link in vivo, increasing and/or maintaining viscosity and resistance to degradation, leading to an improved performance over a longer time period.

In particular the inventive compositions increase the viscosity of the resulting gel whereas prior art hyaluronic acid compounds and compositions, especially in medical and cosmetic applications, have shown decreased viscosity values in vivo after their application and/or unfavorable half life values which require repeated injections. In contrast the inventive compositions achieve increased viscosity and increased half-life, and thus the number of injections can advantageously be reduced.

The inventors have also found that the inventive compositions exhibit an advantageous antioxidant power in the obtained conjugates.

It was surprising that the compositions of the invention, e.g. HA-4Ar as a preferred composition, would lead to increased crosslinking in the hyaluronic acid and with the antioxidant and thus would increase half life and the features of the hyaluronic acid by exposure to in vivo oxidative stress. Usually this leads to a degradation and reduces the applicability in medical and cosmetic applications, however, unexpectedly the compositions according to the invention increases and improves its characteristics under such conditions.

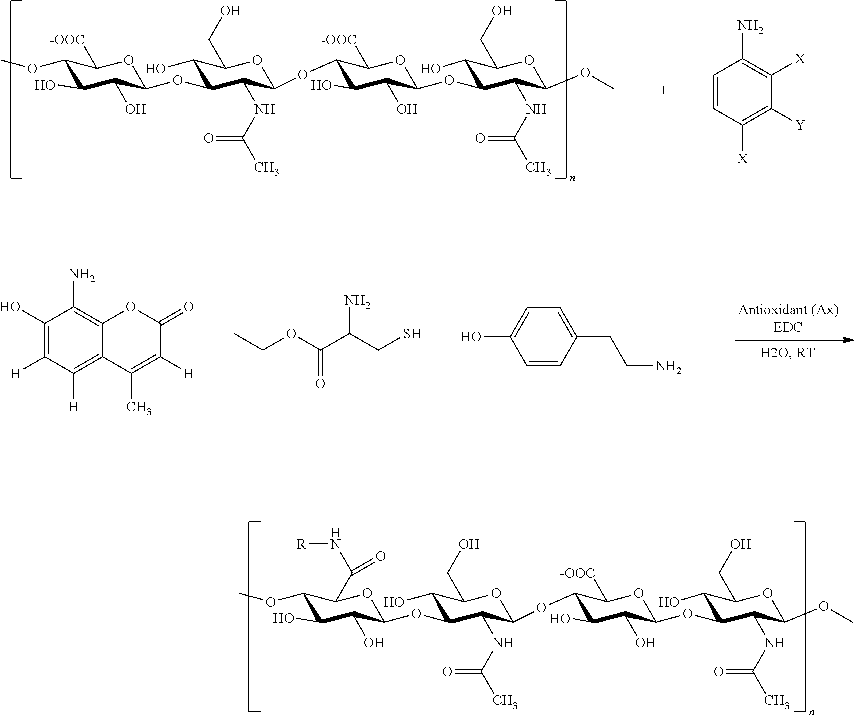

| | Substituant R | | | | | |
|---|---|---|---|---|---|---|
| Moiety | X | Y | Z | AMC | Tyr | EECyst |
| Anilin 2AP 4ASA 5ASA 4AR | H H OH COOH OH | H H COOH OH H | H OH H H OH | 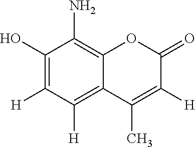 | 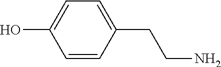 | 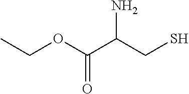 |

In FIG. 1 the synthesis scheme of HA conjugates is depicted.

The grafting percentage and the antioxidant activity of the conjugates are quantified as well as the mass weight the mass weight and the viscosity. The effect of moiety chemical reactivity on grafting percentage obtained is discussed and the antioxidant capacity of the conjugates is measured as well as their degradation profile in an oxidative environment comparable to in-vivo conditions. The most potent conjugates are tested in term of resistance towards hyaluronidase and the most interesting are formulated as a sterile, neutral, isotonic formulation with a viscosity comparable to other viscosupplementation products available on the market. Finally, this formulation will be tested in term of intra-articular biocompatibility in rabbits.

### BRIEF DESCRIPTION OF THE FIGURES

FIG. 1: Synthesis scheme of HA conjugates

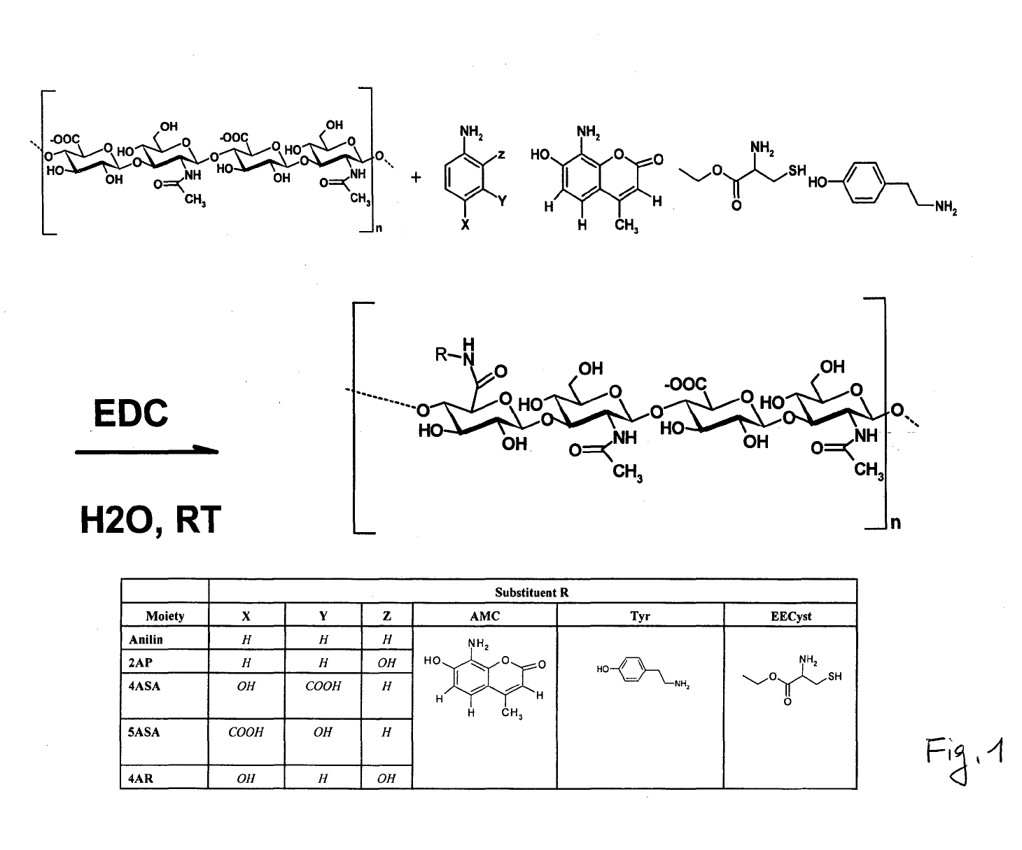

FIG. 2: ¹H-NMR spectra of aniline in D₂O

FIG. 3: Loss viscosity [%] for HA at 1% and the different conjugates in the Weissberger oxidative system compared to viscosity value with copper chloride. n=1.

FIG. 4: Histology pictures, a: Slightly hypertrophic and proliferation of synoviocytes with slight villous hyperplasia. b: Loss of staining visible with Safranin O in superficial zone of hyaline cartilage.

FIG. 5: Echography pictures; A (lateral view) of left side (A2G: nothing injected) and B showing animal 1 left side (A1G: HA-4Ar injected). PAT: patella, FEM: femur, LIQU: liquid, TIB: tibia.

FIG. 6: Picture of femoral articulation of animal 2, left side (A2G: nothing injected) and animal 1 left side (A1G: HA-4Ar injected) after china ink application.

FIG. 7: Synthesis of HA-Antioxidant conjugates (HA-Ax)

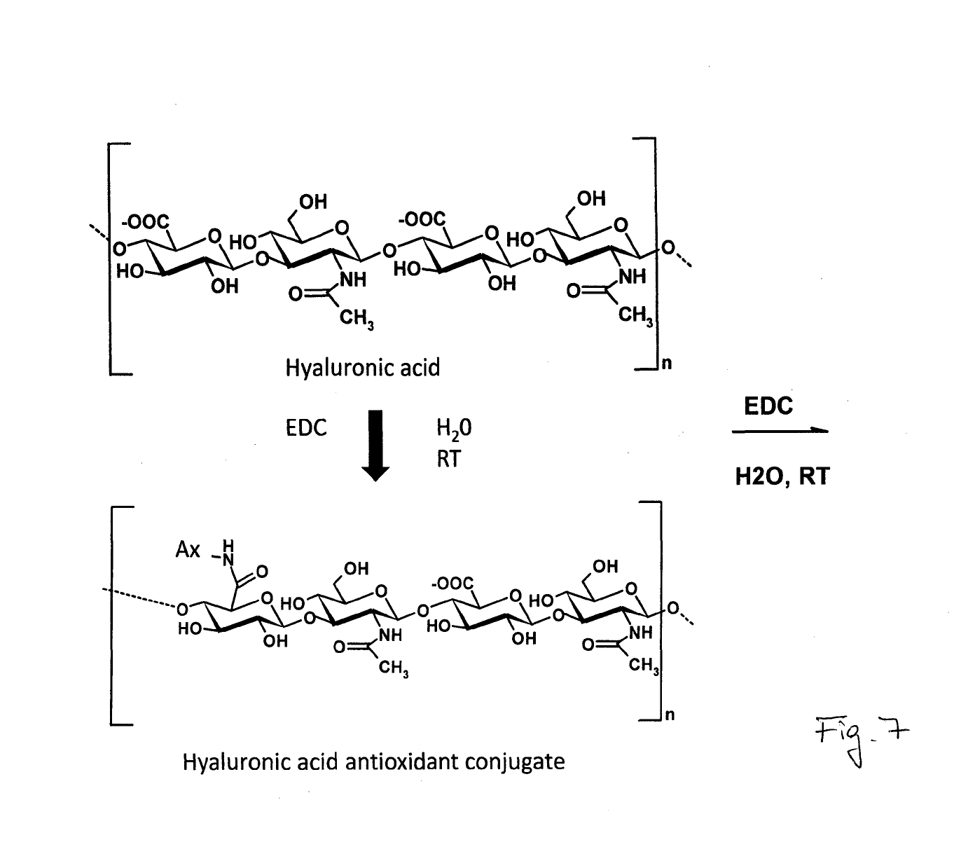

FIG. 8: Oxido-viscosification phenomenon of HA-4AR

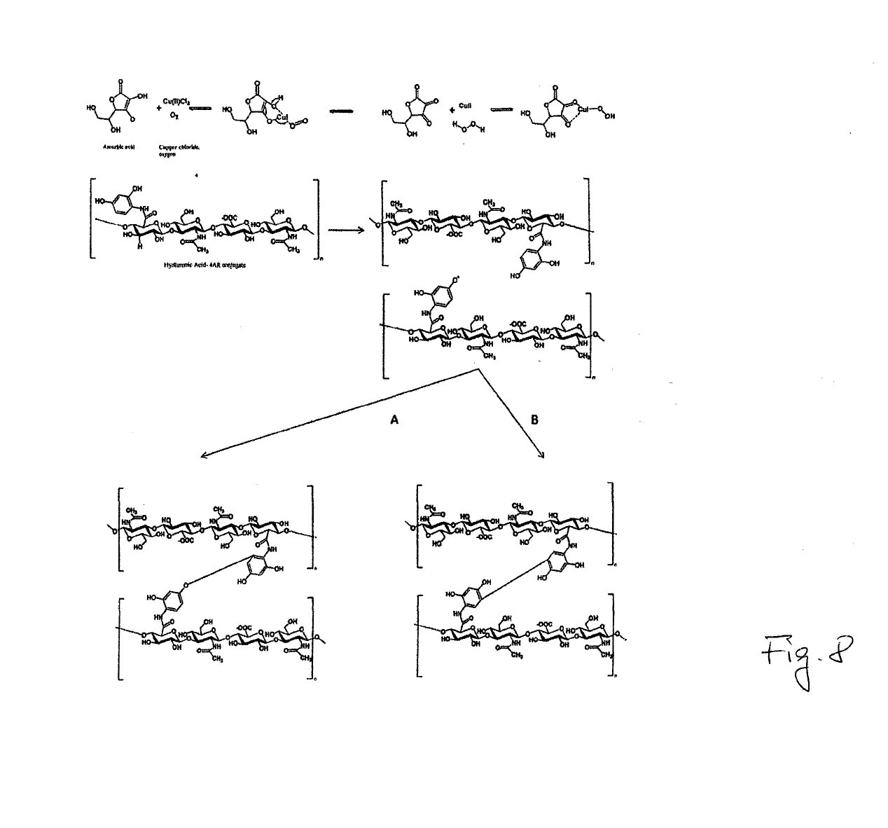

### EXAMPLES

### HA Conjugates Synthesis

The general procedure is to solubilize the maximal concentration of aniline (1 equivalents of COOH: 5.5 mM, 2 equ: 10.9 mM), 2-aminophenol (2 equ), 4ASA (2 equ), 5ASA (1 equ), AMC (1 equ but not totally soluble) or 4AR (1 equ) in 55 to 230 ml water depending on solubility, add 120 mg (10e-4 mM) of HA and allowed to hydrate protected from light under magnetic mixing (500 rpm) until an homogeneous and slightly viscous solution is obtained. The pH of this solution is adjusted to 4.8 and 0.75 equivalent to moiety of EDC is added as a powder (4 mM, 43 mg, 0.75 equ COOH). After EDC addition, a decrease of pH is seen and after around 5 minutes the pH returns to 4.8. In some cases, the reaction mix is passed through a preparative SEC column. For the upscaled reactions, 500 mg of HA are used with the corresponding quantity of antioxidant and EDC and the reaction is performed under mechanical stirring (1100 rpm).

For tyramin, protocol is optimized on the basis of the literature [9]; 100 mg HA are allowed to hydrate, 457 mg tyramin (10 equivalents of COOH) are added and the pH brought to 4.8. 30 mg of NHS (0.1 NHS:EDC) and 504 mg EDC (10 equ COOH) are added and pH is maintained at 4.8 during 15 minutes and then raised to 5.75 and maintained above 7.2 during 24 hours.

To precipitate the obtained product, 0.1 ml of saturated sodium chloride solution is added per ml of purified product obtained. Ethanol (2.3 ml/ml) is added under stirring and the mix is put in the fridge for 1 h and the precipitate is isolated by centrifugation. The precipitate is solubilized in sodium chloride 6% (0.2 ml/ml), precipitated with 0.7 ml/ml ethanol and isolated by centrifugation. Finally, the precipitate is re-suspended in 0.5 ml/ml ethanol and dried under vacuum at 60° C.

For EECyst, the protocol is optimized on the basis of a protocol found in the literature [10]. A solution of HA of 0.4% (m/V) is prepared; 500 mg of HA are hydrated in 125 ml of water and put under mechanical stirring (env. 1400 rpm). The pH of the solution is adjusted to 5.5 with and EDC, NHS are added to have a final concentration of 50 mM (EDC 4.75 equ COOH, 1.0 equ EDC/NHS); 1197.5 mg of EDC and 720 mg of NHS. The pH is then stabilized at 5.5 during 15 minutes and 625 mg of ethylester cystein hydrochloride (27 mM, 2.56 equ COOH) are added and the pH of the mix adjusted to 6.0 during 4 h. After 4 h, the pH of the mix is adjusted to pH 8.0 and an excess of five molar fold (of EEcyst) dithiothreitol (DTT) is added (2075 mg). Finally, the pH is maintained at pH 8.0 during 12 hours. pH of the mix is then adjusted to pH 3.5 and put into dialysis membranes against acidified water (pH 3.5) during 12 hours (1 change of water every 2 hours). Then, HA is precipitated with ethanol; to the 200 ml of reaction mix are added 800 ml of ethanol. The mix is stirred and then centrifuged at 4000 rpm 5 minutes. Once the supernatant removed, the polymer is re-suspended in ethanol and dried under vacuum at 60° C.

### Conjugate Characterization

### Structure Analysis by ¹H-NMR

The obtained conjugates are characterized by ¹H-NMR in D₂O.

### Grafting Semi-Quantification by Spectrophotometry

Grafting percentage is expressed by percent of carboxylate modified and corrected with the native HA Mw. Semi-quantification of the linking is done by spectrophotometry analysis (at 230 nm for aniline and 2-aminophenol, 295 nm for 5ASA, 265 nm for 4ASA, 374 nm for AMC and 276 nm of 4AR and Tyr 275 nm) and expressed in percent of HA carboxylate grafted. Calibration curve in water (6.7e-6 to 9.5e-3 M) is measured in water for each moiety and a conjugate solution of 1 mg/ml is analyzed.

For EECyst, the protocol to dose the total thiol content is based on the literature [11]. A calibration curve with EECyst (2.2-3 mM to 1.1e-1 mM) in water is performed and to each solution 250 ul of NTSB (prepared according to literature [11]) stock solution (1 mg ad 100 ml buffer prepared as follow; 0.2M Tris HCL pH 9.5 with 0.1M Na2SO3 and 3 mM EDTA.) is added and allowed to rest 25 minutes before reading at 412 nm. For sample analysis, three independent solutions at 1 mg/ml in water are submitted to the same procedure as described for calibration.

### Antioxidant Activity Measurement

Method is taken from the literature [12]. A calibration curve is performed in water with a stock solution of DPPH in methanol (1e-4 to 5e-6 M) at 515 nm. 5 mg of antioxidant or conjugate are solubilized in 100 ml of water and to 1.5 ml of the sample 1 ml of stock DPPH solution is added and the mix is allowed to rest at ambient temperature for 60 minutes before measurement of absorbance.

### Viscosity Measurement

0.4 ml of conjugate gel or formulation at different polymer concentration is measured in a logarithmic constant rate mode (10 steps) from 0.1[s⁻¹] to 100.0 [s⁻¹] at 37° C.

### Viscosity Stability in an Oxidative Environment

The same rheological program as described above is used and repeated during 30 minutes. The above protocol is taken from literature [13]; to 0.5 ml of a gel at 1% (m/V) 10 μl of CuCl₂ solution (0.26 mM) is added and the viscosity measured. A second measurement is done with the same procedure with the addition of 10 ul ascorbic acid solution (25 mM).

### Conjugate Formulation

The gel is steam sterilized 10 minutes at 121° C. with an entire cycle of 35 minutes. For the viscosity, gels are measured with the rheological program presented before.

### Intra-Articular Biocompatibility Test

Twelve male New-Zealand rabbits are kept in individual cages 2 weeks after reception before injection. The day of injection, they are anesthetized with Ketamin©/Domitor©, their posterior articulations shaved and disinfected with Vetedine© soap and solution. 200 ul of the selected formulations are injected into the articulations. The rabbit is then revived with Atipam©. The formulations are randomized for rabbits as well as for articulations and injected blindly.

Twice a week, a clinical examination is performed comprising the following points. For the general state; vigilance (at distance from the cage: normal, diminished, sleepy, enhanced), movements (at distance from the cage: normal, diminished), limb ledge (at distance from the cage: yes, no), limp (at distance from the cage: yes, no), hair state: (smooth and shiny, dull, bristling, dirty zones, hairless zones), wound: (yes, no), abnormal secretion: (yes, no), mucosal color: (pink, blade, congested, cyanosed). For the injected joints; swelling (from 0 to 3, 3 being strongest swelling), flow at the injection site (0-3), heat (0-3), palpation pain (from 0 to 3), mobilization pain (0-3), redness (0-3), hematoma (0-3). An echography is also performed under anesthesia comprising the following four views (external lateral, internal lateral and proximal) according to a protocol published in the literature [14] and provides information about the presence of liquid, osteophytes and any severe cartilage erosion. After one month observation, the rabbits are sacrificed with intra-muscular injection of Ketamin©/Domitor© and intra-cardiac Dolethal©. The articulations are dissected and photographed before and after china ink embodiment to assess the state of the rotula, condyl, tibia and trochlea cartilage by a visual analogue scale (VAS) expressed in percentage of the structure degraded and by osteophytis scoring (OS) giving the presence or absence of osteophytes (0: absent, 1: present) [15].

The femoral and tibial parts are fixed in 10% formalin solution and decalcified with Kristenson solution from Chimie-Plus Laboratoire (Denice, France) during several weeks. Samples are cut in two parasagittal sections for medial femoral and tibial condyles (left and right) and then paraffin embedded and sectioned into 4 μm thick slices. Sections were stained with Safranin O-fast green for cartilage. Synovial membranes were fixed in 10% formalin solution, cut in two or three parallel sections, paraffin embedded and sectioned into 4 μm thick slices. Each paraffin block was sectioned in three slides [one stained with hematoxylin, eosin and saffron (HES), and two stained with hematoxylin and eosin (HE)]. Histological features were evaluated in a blinded fashion except for control rabbits. Histological lesions were evaluated and scored according the OARSI histopathology initiative; -recommendations for histological assessments of osteoarthritis in the rabbit (Osteoarthritis and cartilage 18 (2010) S53-S65). Microscopic scoring of cartilage alterations, two sections per condyle were evaluated allowing a semi-quantitative grading. Several parameters were evaluated: Safranin O-fast green staining, structure, chondrocytes density, cluster formation. For microscopic scoring of synovial alterations, three sections per synovial membrane were evaluated, allowing semi-quantitative grading. Several parameters were evaluated: synoviocytes proliferation, synoviocytes hypertrophy, granulocytic infiltration, lymphoplasmacytic infiltration, lymphoplasmacytic aggregates follicles, villous hyperplasia, proliferation of fibroblasts/fibrocytes, proliferation of blood vessels, cartilage/bone detritus presence and hemosiderosis.

### Conjugate Characterization

### ¹H NMR Analysis

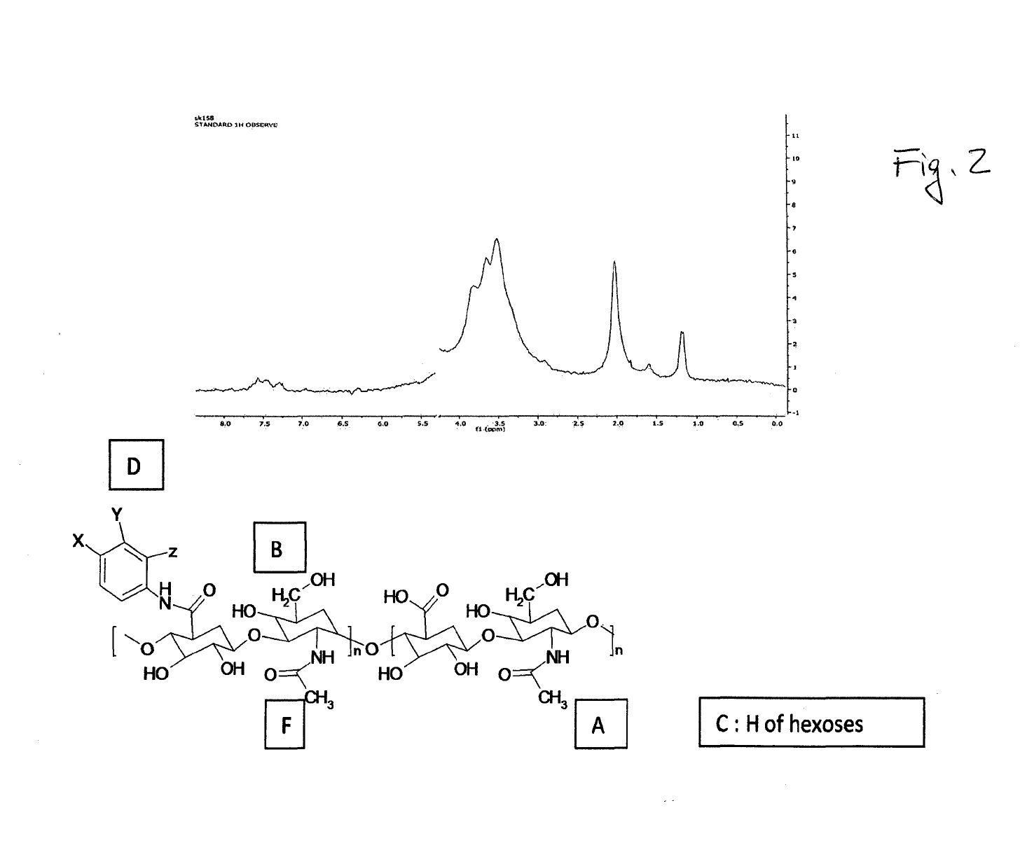

Final products were observed as white to brown fibrous materials totally to slightly soluble in water. Confirmation of the amide formation is done by appearance of peak at 1.5 ppm. Indeed, this peak corresponds to NCH₃ protons shielded by the presence of the grafted moiety [4]. For some conjugates, the protons of the aromatic protons of the moiety are also visible. FIG. 2 shows the ¹H-NMR spectra of aniline in D₂O

TABLE 1
Peak presence in the different conjugates obtained

| | Peak presence | | |
|---|---|---|---|
| | NCH₃ | Aromatics | N-Acylurea * |
| Moiety | (amide, F) | (D) | (E and F) |
| HA | − | | |
| Aniline | + | + | + |
| 2AP | | + | + |
| 4ASA | | + | + |
| 5ASA | | + | + |
| AMC | | − | + |
| 4AR | | − | + |
| Tyr | − | + | + |
| EECyst | + | EEcyst | + |
| | | peaks: + | |

* E is located around 2.3 ppm (H₃CN/—H₂CN) and F near 3.0 ppm (—CH₂)

N-acylurea byproducts are present in all the conjugates. Aromatic protons are not possible to detect for AMC conjugate, this can be explained by the low grafting. For 4AR, it is possible that the aromatic protons are not detected because of an improved resonance. Concerning tyrosine, peak characteristic of amide is not visible, probably due to the fact that the aromatic moiety is not directly linked to HA backbone as for the other aromatic moieties.

### Effect of the Moiety Nature on Grafting Percentage

In order to first optimize reaction conditions, grafting was done with 1 equivalent COOH aniline, taken as the simplest, non-substituted aromatic ring possible to conjugate. Three pH values were tested for reaction in accordance with pKa of aniline (4.5); no pH intervention, pH maintained at 6.5 (pH=pKa+2) and maintained at 8 (pH=pKa+3). Concerning the reaction time, as HA is less degraded at acidic pH (<4) than at basic pH (>11), reaction time is reduced for basic pH [16].

TABLE 2
Linking percentage of aniline on HA
at different pH and reactions times.

| | Time | Anilin |
|---|---|---|
| pH | [h] | [COOH %] |
| NR (5.4/6.0) | 2/4 | 13/1* |
| 6.5 | 2/4 | 14/4* |
| 8.0 | 0.5 | 5* |
| NR (5.0) with 2 equ. | 4 | 25 |

n = 1
NR: pH not regulated.
In brackets: final pH.
*purified by preparative SEC.

Less purification is needed at higher grafting levels. Indeed, the N-acylurea byproducts produced lead to partial to total non-water solubility depending on the concentration. In some cases, SEC purification or dialysis results in water soluble products which still contain side products. Since N-acylurea is a similar size to HA, the conclusion is that the HA side products are more prone to hydrolysis in contact with water than non-grafted HA, leading to partial purification. The final polymer solubility then depends on the side product concentration.

Reaction is as efficient with no pH regulation than at pH 6.5 but at pH 8, there is already need for purification after 30 minutes. Indeed, the amine group of aniline is more nucleophilic at pH 8 to attack the carboxylic acid but, as the pKa of HA amine is 6.5, there is a competition for amide formation, which leads to low reactivity.

At pH not regulated and at pH 6.5, an extended reaction time (4h) leads to lower substitution due to competition with side products formation which is counterbalanced by addition of 30 equivalents of aniline. Nevertheless, increasing aniline concentration by two-fold permits a clear increase of the grafting percentage. In conclusion, it is possible to say that pH of the reaction depends on amine pKa of aniline and HA. Increased reaction time does not affect grafting, but the doubling of aniline concentration leads to increased grafting.

For the antioxidant moieties, the same conditions are tested; 2 h with no pH regulation and the maximal moiety concentration (2AP:7.5, 4ASA 10.5, 5ASA: 15, AMC: 15 and 4AR:15 equivalents). 2AP and 4ASA produce soluble conjugates but 5ASA, AMC and 4AR need pH 8.0 for at least 30 minutes to form a soluble product. For tyramin and EECyst, conditions are difficult to compare as there is NHS present and the ratio of EDC is different.

TABLE 3
Linking percentage of obtained conjugates at optimized conditions.

| | | | | | | | Mw |
|---|---|---|---|---|---|---|---|
| | | Amine | | | | Grafting | loss |
| | Amine | activation/ | | | Timing | [% COOH] | [%] |
| Moiety | pKA | deactivation | Equivalents | pH | [h] | n = 1 | n = 2 |
| Anilin | 4.5 | —/— | 2 | NR | 4 | 25 | 0 |
| | | | | (5.0) | | | |
| 2AP | 4.7 | +(1)/— | 2 | NR | 4 | 44 | 64 |
| | | | | (5.0) | | | |
| 4ASA* | 4.6 | —/— | 2 | NR | 2/4/0.5 | —/—/1 | —/—/33 |
| | | | | (5.6/5.2)/8 | | | |
| 5ASA | 6.0 | +(1)/— | 1 | 8.0 | 0.5/1 | 2/2 | 0/55 |
| AMC | 2.4 | —/— | 1 | 8.0 | 0.5/1 | 0.1/0.1 | 45/22 |
| 4AR | 5.8 | +(2)/— | 1 | 8.0 | 0.5/1 | 14/57 | 23/ |
| Tyr | 10.8 | Not applicable | 1 | 5.75-7.5 | 24 | 5 | 66 |
| EECyst | 10.8 | Not applicable | 2.5 | 6.0 | 4 | 18 | 32 |

In brackets: final pH.
*purified by preparative SEC.
—: not purifiable.
Brackets: number of amine activation/deactivation

The order of reactivity is the following: 2AP>aniline>EECyst>Tyr>4AR>5ASA>AMC and 4ASA. AMC, aniline, 2AP and 4ASA present pKa's lower than 5, resulting in favourable amidation at pH around 5.0 with no external pH regulation. 2AP shows the highest grafting percentage even with its low water solubility resulting from activation of the amine by the hydroxyl in the ortho-position. Aniline has no substituent acting as activators or inactivators of the amine but its high water solubility is favouring amide formation. 4ASA presents the lowest grafting of this group of compounds because it is inactivated, has low water solubility and a carboxyl present (activated by EDC). Indeed, the reaction with 15 equ for 30 minutes at pH 8.0 leads to grafting percentage of only 1% without purification meaning that for moieties with low pKa and a inactivated amine, it is more favorable to make the reaction at pH=pKa+2 than at pH=pKa.

4AR and 5ASA present higher pKA than the three first compounds resulting in the need to perform amidation at a higher pH. Indeed, the amine is more nucleophilic at pH equal or above pKa. In this group, 4AR presents the highest grafting percentage resulting from the double activation by ortho- and para-hydroxyl groups. The decreased grafting of 5ASA can be explained by carboxyl presence and singlee activation of the amine by the para hydroxyl group. Concerning AMC, the solubility is low and the pKa of the alcohol group is 7.8 (Calculated with ACD Lab). At pH 8, it is charged, leading to decreased reactivity. Reactivity for tyramin and EEcyst are difficult to compare but given they both have high pKa and are non-aromatic, they exhibit a high reactivity toward amidation. In an upscale of 4AR conjugate preparation from 120 mg to 500 mg, it has been observed that rotation is the key factor to obtain a soluble conjugate but that decreased grafting percentages are obtained for the same reaction time. Extended reaction time fails to substantially improve grafting (2 h: 14% grafting and 35% Mw loss and 4 h 16% grafting and 31% Mw loss).

As a conclusion, it is possible to say that pH of reaction strictly depends on pKA and that higher grafting is obtained with molecules with pKa close to 5.0. Indeed, EDC is more reactive between pH 3.5-4.5 than at higher pH [17]. Concerning grafting percentage, a balance between amine activation, water solubility and carboxyl presence will be determined by one skilled in the art. At optimized reaction conditions, results are reproducible.

Conjugates exhibit mass weight loss which cannot be attributed to reaction pH, timing conditions nor grafting percentage. Schanté et al. explains that the different derivatives are not subjected to the same fragmentation of the HA backbone during reaction [18].

### Antioxidant Activity

Table 4 presents the conjugates, grafting percentage and DPPH % after 60 minutes, characterising of the antioxidant activity of the conjugates. The lower this value is, the more efficiently the conjugate stabilize the DPPH radical, the more the conjugate is antioxidant.

TABLE 4
Grafting percentage and DPPH percentage for the different
conjugates and free moieties

| | | | Number | DPPH for | | |
|---|---|---|---|---|---|---|
| | | | of | moiety | Grafting | |
| | | | Hydrogen | alone | [% | DPPH |
| | Amine | Amine | sharing | [%] ± SD, | COOH], | [%] ± SD, |
| Moiety | pKA | activation/deactivation | groups* | n = 3 | n = 1 | n = 3 |
| HA | 6.5 | Not applicable | Not | 0 | 0 | 93 |
| | | | applicable | | | |
| Aniline | 4.5 | —/— | 1/0 | 36 | 25 | 95 |
| 2AP | 4.7 | +(1)/— | 2/1 | 17 | 44 | 82 |
| 4ASA | 4.6 | —/— | 3/2 | 27 | 6 | 96 |
| 5ASA | 6.0 | +(1)/— | 3/2 | 34 | 2 | 67 |
| AMC | 2.44 | —/— | 3/2 | 87 | 0.1 | 61 |
| 4AR | 5.8 | +(2)/— | 3/2 | 28 | 14/57 | 71/37 |
| Tyr | 10.8 | Not applicable | 2/1 | 85 | 5 | 92 |
| EEcyst | 10.8 | Not applicable | 2/1 | 6 | 18 | 41 |

*Not grafted/grafted including NH₂, OH and COOH

The order of DPPH stabilization is the following for the free moieties: EECyst>2AP>Anilin>4AR>4ASA>5ASA>Tyr>AMC>HA. It is important to note that those values comprise the free amine which is not yet linked to HA.

HA itself does not exhibit any antioxidant properties. The antioxidant capacity of the moieties is not related to the number of hydrogen sharing groups. Carboxylic group diminishes antioxidant capacity and as mentioned in the literature [19], compounds with a hydroxyl in ortho-position of amine (2AP and 4AR) show increased antioxidant capacity. Surprisingly, aniline exhibits a high antioxidant capacity although it has no hydrogen donor group. AMC does not exhibit a high antioxidant capacity even though it has a bi-cyclic structure with an ortho-positioned of the amine hydroxyl group. It has been reported in the literature that for more bi-cyclic structures, hydrogen sharing groups is not an important parameter for antioxidant activity [20].

Concerning the conjugates, the order of antioxidant activity is the following: 4AR>EECyst>AMC>5ASA>2AP>Anilin>Tyr>4ASA. 2AP, aniline, tyramin and 4ASA are the least antioxidant of the conjugates, mainly due to the loss of hydrogen sharing groups

4AR, 5ASA and AMC retain 2 hydrogen sharing groups after grafting and 4AR shows the highest activity as it is also the more grafted of those conjugates. It is important to see that ethylestercystein presents a much lower grafting percentage than 4Ar but an interesting antioxidant activity due to its free thiol and that the conjugate with AMC, even if the grafting is very low shows an interesting antioxidant activity.

### Viscosity Maintenance in an Oxidative Environment

The following graphs present the viscosity in function of the time of those conjugates which presented a DPPH percentage above 70%, when exposed to Weissberger's oxidative system.

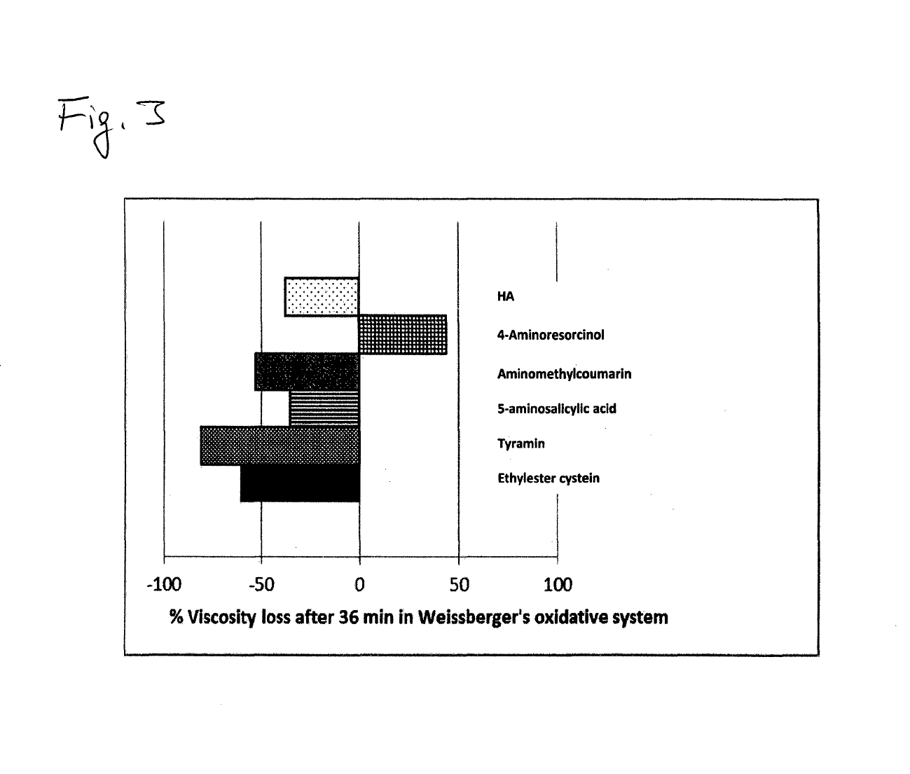

FIG. 3 shows the loss of viscosity [%] for HA at 1% and the different conjugates in the Weissberger oxidative system compared to viscosity value with copper chloride. n=1.

HA in contact with copper presents a stable viscosity but after ascorbic acid addition, as hydrogen peroxide is generated, degradation occurs. The order of degradation is the following: Tyr>AMC>EECyst>HA>5ASA>>4AR. Tyr and AMC and EECyst conjugates are more degraded than HA itself, probably by a pro-oxidant effect of antioxidant moiety [21]. 5ASA conjugate shows a small protection against oxidative stress.

The antioxidant activity measured by the DPPH test does not correlate with the viscosity measurement in oxidative environment due to the two different mechanisms; DPPH radical stabilization and copper complexation and/or H₂O₂ stabilization for Weissberger oxidative system.

Surprisingly, 4AR gains 40% of viscosity during the 36 minutes of measurement compared to the other conjugates which lose viscosity during the measurement.

### Conjugate Formulation

The target characteristics are a viscosity after heat sterilization of around 1000 [mPas], neutrality and isotonicity to plasma (around 280 [mOsmol]) in common with state of the art commercial formulations such as Ostenil©.

The following formulation fulfills all those characteristics: 2.7% (m/V) 4AR conjugate (16% grafting and 13% Mw loss), 0.85% NaCl, 0.24% Na₂HPO₄, 0.02% NaH₂PO₄ with a pH of 7.2 and a viscosity of 824.3±55.9 [mPas]. It was verified by SEC that the antioxidant is still linked to the polymer after steam sterilization. SEC analysis of the steam sterilized formulation presents a UV peak which still matches the MALLS peak (MW loss: 55%), meaning that that antioxidant remains bound to the polymer.

### Intra-Articular Biocompatibility in Rabbit

The above mentioned formulations were tested in term of intra-articular biocompatibility in the rabbit model; Conjugated HA linked to 4AR (n=4), unconjugated HA mixed with 4AR (n=4), 4AR (n=4) and as controls; physiological serum (n=2), untreated (n=2) and Ostenil© (n=4).

Characteristics of the following formulations are presented in table 5.

TABLE 5
Content of formulations with pH and viscosity obtained after
steam sterilization. Percentages are mass per volume.

| | | | | Buffer | | | Viscosity |
|---|---|---|---|---|---|---|---|
| | Polymer | 4AR | NaCl | Na₂HPO₄ | NaH₂PO₄ | | [mPas] ± SD |
| Formulation | [%]* | [%] | [%] | [%] | [%] | pH | n = 3 |
| HA-4AR | 2.7 | 0 | 0.85 | 0.24 | 0.002 | 7.18 | 824.3 ± 55.9 |
| HA + 4AR | | 0.005 | | | | 6.95 | 1015.3 ± 36.1 |
| (16% graft) | | | | | | | |
| 4AR | 0 | | | | | 7.25 | 0 |

*HA or 4Ar conjugate

Note that the antioxidant concentration present in the blank formulation (4AR mixed with HA) is lower than the concentration present in the antioxidant grafted HA. Indeed, linking of 4AR onto the HA backbone increases 4AR solubility substantially.

Concerning clinical exam, echography, macroscopy and histology no significant differences between control and treated rabbits was observed.

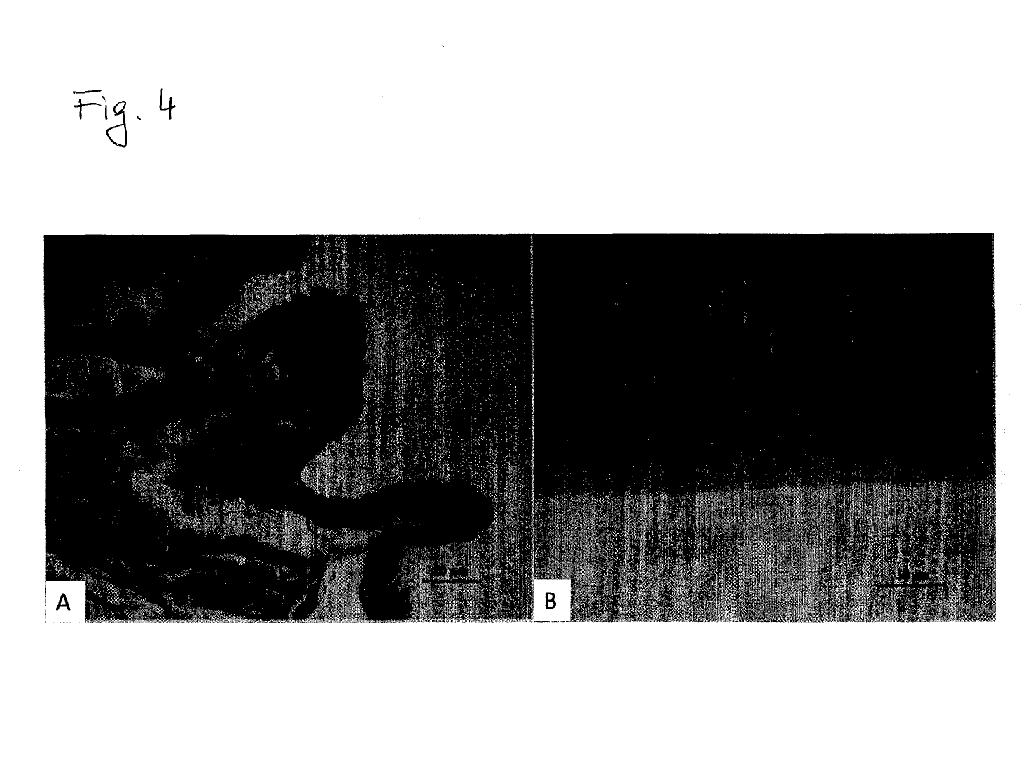

FIG. 4 shows Histology, a: Slightly hypertrophic and proliferation of synoviocytes with slight villous hyperplasia. b: Loss of staining visible with Safranin O in superficial zone of hyaline cartilage.

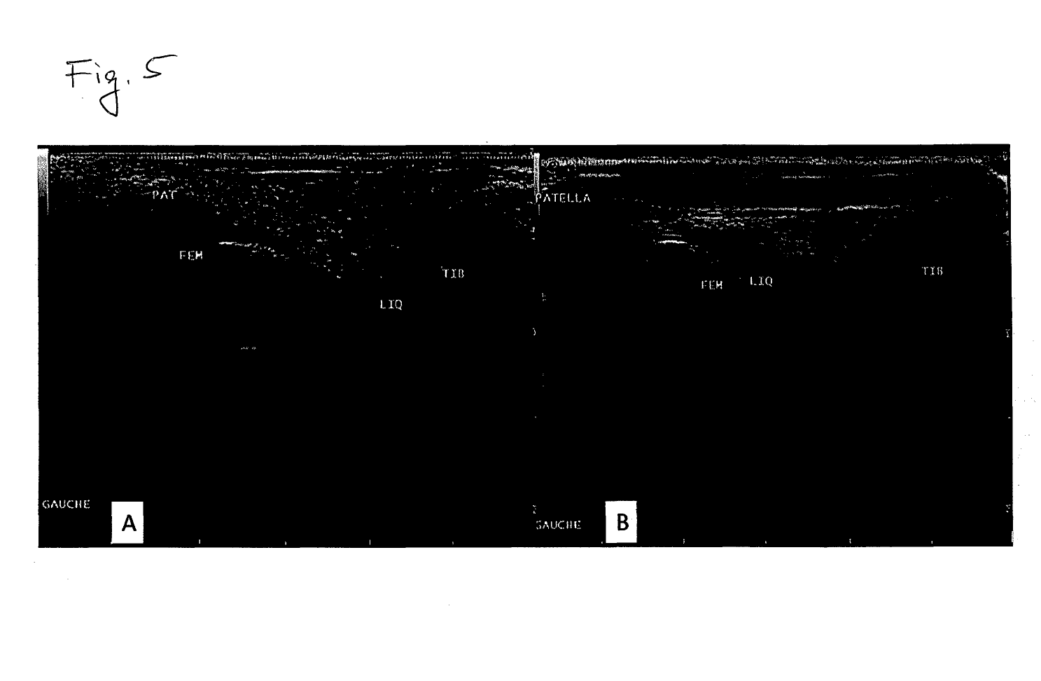

FIG. 5 shows an Echography; A (lateral view) of left side (A2G: nothing injected) and B showing animal 1 left side (A1G: HA-4Ar injected). PAT: patella, FEM: femur, LIQU: liquid, TIB: tibia.

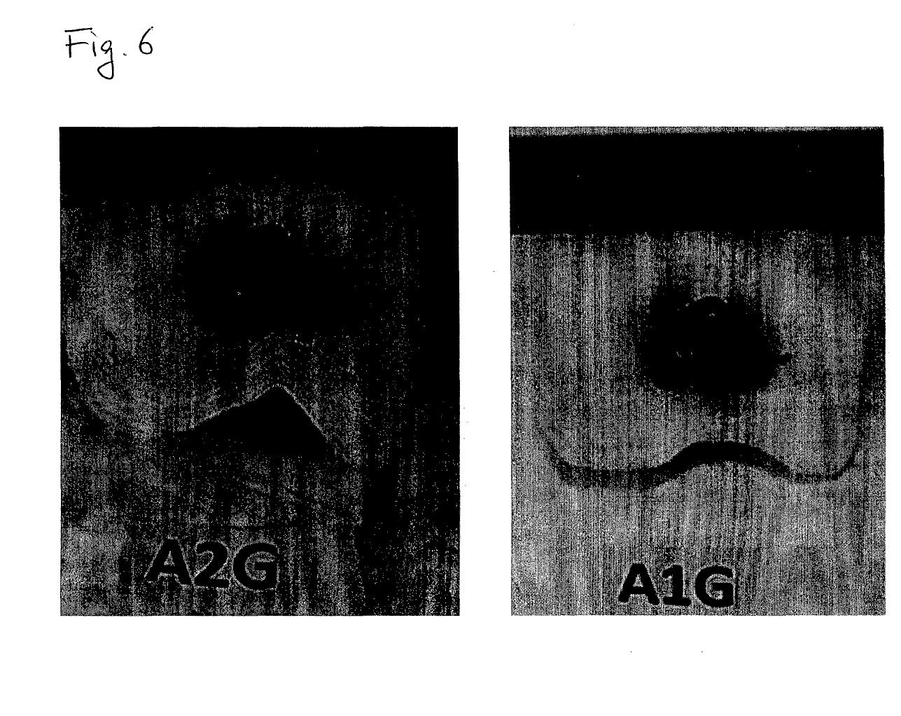

FIG. 6 shows pictures of femoral articulation of animal 2, left side (A2G: nothing injected) and animal 1 left side (A1G: HA-4AR injected) after china ink application.

TABLE 6
VAS score, osteophyte score (OS) as well as signs of synovial
membrane inflammation or fibrosis and meniscus lesions.
VAS: percentage of structure degraded
OS: 0 if absent and 1 if small osteophyte present.

| | | | Condyl | | Tibia | | | | | | |
|---|---|---|---|---|---|---|---|---|---|---|---|
| | Rotula | | (int/ext) | | (int/ext) | | Trochlea | | Synvovial | | Meniscus |
| | VAS score | OS | VAS score | OS | VAS score | OS | VAS score | OS | membrane | | lesion |
| Formulation | [%] | [%] | [%] | [%] | [%] | [%] | [%] | [%] | Inflamation | Fibrosis | (int/ext) |
| HA-Ax, | 0 | 0 | 0/0 | 1*/0 | 0-10° /0-10** | 0 | 0 | 1* | 0 | 0 | 0 |
| n = 4 | | | | | | | | | | | |
| HA + Ax, | 0 | 0 | 0/0 | 0/0 | 0-10***/0-10*** | 0 | 0 | 0 | 0 | 0 | 0 |
| n = 4 | | | | | | | | | | | |
| Nothing, | 0 | 0 | 0/0 | 0/0 | 0-10° /0-10* | 0 | 0 | 0 | 0 | 0 | 0 |
| n = 2 | | | | | | | | | | | |
| Ax, n = 4 | 0 | 0 | 0/0 | 0/0 | 0***/0*** | 0 | 0-10* | 0 | 0 | 0 | 0 |
| Serum | 0 | 0 | 0/0 | 0/0 | 0-10*/0-10* | 0 | 0-10 | 1* | 0 | 0 | 0 |
| ph, n = 2 | | | | | | | | | | | |
| Ostenil, | 0 | 0 | 0/0 | 1*/0 | 0-10°/0° | 0 | 0-10* | 1* | 0 | 0 | 0 |
| n = 4 | | | | | | | | | | | |

*1animal touched,
**2 animals,
°all the animals.

In the view of those tests, it is possible to say that the 4AR conjugate formulation tested exhibits noinduced inflammation nor arthrosis after one month. Although free 4AR is a potent cytotoxic agent [22], no sign of non-biocompatibility could be shown in the tested conditions.

Examples of antioxidants are depicted below:

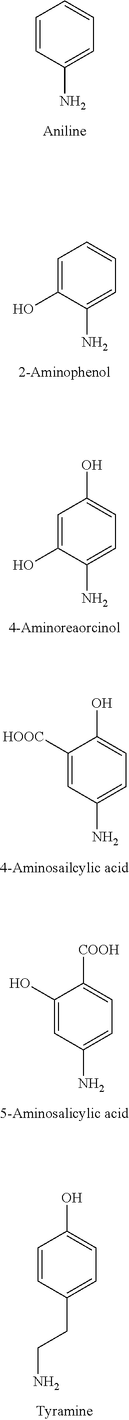 [SMILES: Nc1ccccc1 ; Nc1ccccc1O ; Nc1ccc(O)cc1O ; Nc1ccc(O)c(C(=O)O)c1 ; Nc1ccc(C(=O)O)c(O)c1 ; NCCc1ccc(O)cc1]

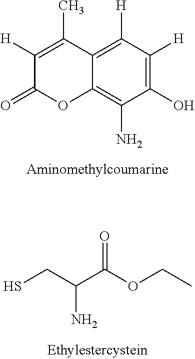

Antioxidant conjugates of hyaluronic acid according to the invention can be synthesized by an one step EDC chemistry in water as proven by ¹H-NMR and SEC-UV-MALLS measurements. Reaction conditions have to be adapted to the particular compounds used and can be optimized by one skilled in the art in order to obtain reproducible totally water soluble products.

In conditions favouring high grafting efficiency, the less purification steps are needed. To obtain improved grafting efficiency, a number of factors concerning moiety are important, these include pKa, water solubility, amine activation and carboxyl group presence. Molecules having a pKa of the amine close to 5.0 are easier to graft as EDC reactivity as well as HA stability are optimal at this pH. All the conjugates show mass weight values above 500 KDA.

Antioxidant activity is conserved after grafting and antioxidant activity of the conjugates depends on grafting percentage and on the number of H donor groups still available after amide formation.

The preferred embodiment of the invention comprising 4AR conjugate exhibits enhanced viscosity in an oxidative environment and is stable under steam sterilization and shows complete biocompatibility after a single injection and one month evaluation. Such conjugates have interesting characteristics for cosmetic and pharmaceutical use.

### LITERATURE

1. Hinton R., M. R., Davis A., and Thomas S., Osteoarthritis: Diagnosis and Therapeutic Considerations. American family physician, 2002. 65(5).
2. Bellamy N, C. J., Welch V, Gee T L, Bourne R, Wells G A., Viscosupplementation for the treatment of osteoarthritis of the knee. Cochrane Database of Systematic Reviews 2006. 2.
3. Jackson, D. W. and T. M. Simon, Intra-articular distribution and residence time of Hylan A and B: a study in the goat knee. Osteoarthritis and Cartilage, 2006. 14(12).
4. Ponedel'kina, I. Y. and V. N. Odinokov, Modifications of hyaluronic acid with aromatic amino acids. Russian Journal of Bioorganic Chemistry 2005. 31(1).
5. Shu, X. Z., Disulfide Cross-Linked Hyaluronan Hydrogels. Biomacromolecules, 2002. 3.
6. Vazquez-Torres, A., Redox active thio/sensors of oxidative and nitrosative stress. Antioxid Redox Signal, 2012. 17(9): p. 1201-14.
7. Tyagi, Y. K., Synthesis of novel amino and acetyl amino-4-methylcoumarins and evaluation of their antioxidant activity. European Journal of Medicinal Chemistry, 2005. 40.
8. Pouyani, T., Solid-state NMR of N-Acylureas Derived from the Reaction of Hyaluronic Acid with Isotopically-Labeled Carbodiimides. J. AM. Chem. Soc, 1992. 114.
9. Darr, Synthesis and characterization of tyramine-based hyaluronan hydrogels. 3 Mater Sci: Mater Med, 2008. 20.
10. Kafedjiiski, K., Synthesis and in vitro evaluation of thiolated hyaluronic acid for mucoadhesive drug delivery. International Journal of Pharmaceutics, 2007. 343.
11. Thannhauser, T. W., Y. Konishi, and H. A. Scheraga, Sensitive quantitative analysis of disulfide bonds in polypeptides and proteins. Analytical Biochemistry, 1984. 138(1): p. 181-8.
12. Popovici, Evaluation de l'activité antioxydant des composés phénoliques par la réactivité avec le radical libre DPPH. Revue de Génie Industriel, 2009. 4.
13. Valachova, K., Degradation of High-Molar-Mass Hyaluronan by Ascorbate plus Cupric Ions: Effects of d-Penicillamine Addition. CHEMISTRY & BIODIVERSITY, 2009. 6.
14. Boulocher, C., et al., Knee joint ultrasonography of the ACLT rabbit experimental model of osteoarthritis: relevance and effectiveness in detecting meniscal lesions. Osteoarthritis Cartilage, 2008. 16(4): p. 470-9.
15. Yoshioka, M., et al., Characterization of a model of osteoarthritis in the rabbit knee. Osteoarthritis Cartilage, 1996. 4(2): p. 87-98.
16. Maleki, A., Effect of pH on the Behavior of Hyaluronic Acid in Dilute and Semidilute Aqueous Solutions. Macromolecular Symposia, 2008. 274: p. 131-140.
17. Nakajima, N. and I. Yoshita, Mechanism of Amide Formation by Carbodiimide for Bioconjugation in Aqueous Media. Bioconjugate Chem., 1995. 6(1).
18. Schanté, C. E., Improvement of hyaluronic acid enzymatic stability by the grafting of amino-acids. Carbohydrate research, 2012. 87.
19. Chen, W., The ortho hydroxy-amino group: Another choice for synthesizing novel antioxidants. Bioorganic & Medicinal Chemistry Letters, 2006. 16.
20. Lien, E., Quantitative structure-activity relationship analysis of phenolic antioxidants Free Radical Biology & Medicine, 1998. 26: p. 285-294.
21. Valachova, K., et al., Ascorbate and Cu(II)-induced oxidative degradation of high-molar-mass hyaluronan. Pro-and antioxidative effects of some thiols. Neuro Endocrinol Lett, 2010. 31 Suppl 2: p. 101-4.
22. Larget, R., et al., Synthesis of novel orthoalkylaminophenol derivatives as potent neuroprotective agents in vitro. Bioorganic & Medicinal Chemistry Letters, 1999. 9(20): p. 2929-2934

1. A composition comprising a hyaluronic acid and an antioxidant.

2. The composition of claim 1, comprising a hyaluronic acid and an antioxidant wherein the hyaluronic acid is conjugated with an antioxidant.

3. The composition of claim 1, comprising a hyaluronic acid and an antioxidant wherein the hyaluronic acid is conjugated with an antioxidant and/or wherein the hyaluronic acid is crosslinked.

4. The composition of claim 1, comprising a hyaluronic acid and an antioxidant wherein the hyaluronic acid is conjugated with an antioxidant and/or wherein the hyaluronic acid is crosslinked wherein the crosslinking occurs in vivo.

5. The composition according to claim 1, wherein the hyaluronic acid has one or more residues which can crosslink in an oxidative system.

6. The composition according to claim 1, wherein the hyaluronic acid has one or more residues which can crosslink in an oxidative system and whereby viscosification is enhanced.

7. The composition according to claim 1, wherein the antioxidant is selected from the group consisting of aniline, 5-aminosylicylic acid, aminomethylcoumarine, 4-aminoresorcinol and ethylester cystein.

8. The composition according to claim 1, wherein the viscosity is increased due to an oxidative environment.

9. The composition according to claim 1, wherein the functionality is extended over time.

10. The composition according to claim 1, wherein the composition is biocompatible.

11. The composition according to claim 1, wherein the composition is not significantly more toxic than hyaluronic acid per se.

12.-14. (canceled)

15. A method of treating arthritis, joint pathologies, articular diseases, eye pathologies, skin treatment, or tissue regeneration in a subject comprising applying a composition according to claim 1 to a subject in need thereof.

16.-18. (canceled)

19. The method according to claim 15 wherein the composition is applied alone or in combination with any suitable adjuvants intradermally or subdermally.

20. The method of claim 15, wherein the composition is applied topically, i.v., i.m., s.c., into a joint by injection or into an eye by injection.

21. The method of claim 15, wherein the method is for an aesthetic or cosmetic application.

22. A method for skin treatment in a subject comprising administration of a composition of claim 1 to the subject.
